(19) United States
(12) Patent Application Publication
(10) Pub. No.: US 2026/0139288 A1
(43) Pub. Date: May 21, 2026

(54) 13-CIS RETINOIC ACID AND NEURODEVELOPMENT DISORDERS

(71) Applicant: Joel Steven Goldberg, Hillsborough, NC (US)

(72) Inventor: Joel Steven Goldberg, Hillsborough, NC (US)

(21) Appl. No.: 19/452,630

(22) Filed: Jan. 19, 2026

(51) Int. Cl. C12Q 1/26; G01N 33/82

(52) U.S. Cl. CPC C12Q 1/26; G01N 33/82; G01N 2800/368

(57) ABSTRACT

13-cis retinoic acid (13-cis) is a potent teratogen that crosses the placenta and exists in equilibrium with 13-trans retinoic acid (13-trans). Compared to 13-trans, 13-cis is preferentially concentrated in the cell nucleus where it is isomerized to teratogenic 13-trans. In pregnant mothers the plasma 13-cis concentration is highest in the first 8-12 weeks of pregnancy. Neurocognitive impairment even in the absence of any physical defects has been reported to be 30%-60% of children exposed to Isotretinoin (13-cis) during the prenatal period. Genetic variability of CYP26A1 and/or CYP26B1 may be passed onto the fetus and is probably associated with increased maternal and fetal retinoid concentrations. Beta carotene crosses the placenta but may be metabolized and/or stored by the fetus differently than in adults. Measuring maternal retinoids and CYP26A1 and CYP26B1 activities may provide insight into the steep rise in neurodevelopment disorders not explained by improvement in diagnosis.

### CROSS-REFERENCE TO RELATED APPLICATIONS

This application claims the benefit of U.S. application Ser. No. 19/415,487 which is incorporated by reference.

### FEDERALLY SPONSORED RESEARCH AND DEVELOPMENT

Not applicable

### BACKGROUND OF THE INVENTION

Neurodevelopmental disorders encompass a range of conditions, including intellectual disability, attention-deficit/hyperactivity disorder, motor and communication disorders, and autism spectrum disorder (ASD). While the increasing prevalence of ASD is often attributed to improved diagnostic practices, there is skepticism that other factors may also contribute. Both environmental and genetic influences may play a role in the development of these disorders. This invention focuses on the impact of 13-cis retinoic acid (13-cis), a known potent teratogen, on fetal neurodevelopment.

### Prevalence of ASD in France and Iceland

The prevalence of ASD is notably low in France, which may be due to differences in diagnostic practices compared to other developed countries. France does not routinely recommend prenatal vitamin A supplementation, nor does it mandate fortification of staple foods with vitamin A. In contrast, Iceland reports one of the highest ASD incidences among Nordic countries, where maternal supplementation with beta carotene and preformed vitamin A is common, alongside consumption of cod liver oil and animal liver. However, food fortification with vitamin A is not standard in Iceland. These observations suggest that excessive fetal exposure to retinoids may be associated with ASD risk. Paradoxically, vitamin A supplementation in children may help restore disrupted retinoid neurodevelopmental pathways, potentially improving ASD symptoms.

### General Considerations of 13-Cis

In humans, maternal retinoid levels are tightly regulated, and both deficiency and excess can adversely affect fetal development. 13-cis, considered the most potent retinoid teratogen, exists in a cis-trans equilibrium, though the precise ratio in the human fetus is not well established. In adults, the reported ratio of 13-trans retinoid acid (13-trans) to 13-cis range is quite variable but many studies report near equal ratios. In pregnant mothers the plasma 13-cis concentration is highest in the first 8-12 weeks of pregnancy and declines thereafter. Retinoic acids cross the placenta and nuclear membrane, interacting with retinoid receptors on chromosomes. Compared to 13-trans, 13-cis preferentially accumulates in the cell nucleus, where it is isomerized to 13-trans and binds to gene-regulating retinoid receptors, including retinoic acid receptors (RAR) and retinoid X receptors (RXR). RARs and RXRs form heterodimers that bind to retinoic acid response elements (RAREs), thereby regulating transcription of genes essential for central nervous system (CNS) development. Neurocognitive impairment even in the absence of any physical defects has been reported to be 30%-60% of children exposed to 13-cis during the prenatal period.

The concentration of 13-cis retinoic acid within the cell nucleus is not due to increased lipophilicity but is likely a result of reduced protein binding with retinol-binding proteins and cellular retinoic acid-binding proteins in the cytosol, compared to 13-trans. Additionally, 13-cis has a significantly longer half-life than 13-trans, indicating that cellular exposure to 13-cis is sustained over time. In the plasma after systemic administration, 13-cis undergoes rapid in vivo oxidative metabolism to 13-cis-4-oxoretinoic acid. In summary, the high teratogenic activity of 13-cis in humans may be related to slow elimination of 13-cis, to metabolism to the 4-oxo-derivative, to increased placental transfer, to continuous isomerization and significant exposure of the target tissue to 13-trans; and to lack of binding to cytoplasmic retinoid binding proteins that could possibly result in ready access to the nucleus. This invention considers the genetic variability of cytochromes CYP26A1 and/or CYP26B1 the major retinoid metabolic enzymes as a cause of 13-cis teratogenic activity.

### Enzyme Regulation of 13-Cis

Beta-carotene 15,15′-dioxygenase (BCO1) is the key enzyme responsible for converting beta carotene into retinoic acids, with retinoid levels providing inhibitory feedback on its activity. Genetic variations typically result in decreased BCO1 activity and no increased activity. Beta-Carotene Oxygenase 2(BCO2), a mitochondrial enzyme, catalyzes the asymmetric cleavage of carotenoids at the 9′,10′double bond, producing apocarotenoids such as β-apo-10′-carotenal and ionone derivatives. Unlike BCO1, BCO2 does not directly generate vitamin A. Instead, BCO2 regulates mitochondrial carotenoid metabolism, prevents excessive carotenoid accumulation, and produces signaling molecules essential for mitochondrial and metabolic health.

In humans, retinoids are primarily metabolized by cytochromes CYP26A1 and CYP26B1. The function of CYP26C1 is less understood and is likely secondary to CYP26A1 and CYP26B1. Genetic variations in these cytochromes are associated with decreased retinoid metabolism with increased retinoid levels and not increased metabolism. Elevated systemic and cytosolic levels of the less teratogenic 13-trans can lead to increased concentrations of the highly teratogenic 13-cis, unless regulated by unknown isomerase mechanisms or changes in protein binding.

The total retinoic acid exposure in the fetus results from both retinoids and beta carotene crossing the placenta, combined with fetal regulatory mechanisms. Retinoids are primarily stored as esters in the fetal liver or metabolized by CYP26A1 and/or CYP26B1, with genetic variability in these enzymes potentially inherited from the mother. In the fetus, beta carotene may be converted to retinoic acid by BCO1 or stored in fat. Notably, neonatal non-brown fat is often observed as white by neonatal surgeons, suggesting fetal beta carotene storage differs from adults and may be largely converted to retinoids. Umbilical cord concentrations of beta carotene are reported to be over twenty times lower than maternal levels, implying fetal metabolism, placental gating, or non-fat storage of beta carotene, or a combination of these factors. However, separate measurements of arterial and venous concentrations are lacking in the literature, and such data could clarify the fate of beta carotene in the fetus.

### PRIOR ART

Goldberg, J. S. (2012). Monitoring Maternal Beta Carotene and Retinol Consumption May Decrease the Incidence of Neurodevelopmental Disorders in Offspring. Clinical Medicine Insights: Reproductive Health, 6, 1-8.

### BRIEF SUMMARY OF THE INVENTION

Although the sharp increase in ASD diagnoses is often attributed to greater diagnostic awareness, there is ongoing skepticism that environmental and genetic factors may also play a role. Understanding these factors could help prevent neurodevelopmental disorders. 13-cis is a potent teratogen present in both the mother and fetus in a dynamic equilibrium. In pregnant mothers the plasma 13-cis concentration is highest in the first 8-12 weeks of pregnancy and declines thereafter. Neurocognitive impairment even in the absence of any physical defects has been reported to be 30%-60% of children exposed to 13-cis during the prenatal period. Genetic variability in the maternal CYP26A1 and CYP26B1 cytochromes that are key enzymes for retinoid metabolism can be inherited by the fetus, and such nucleotide changes are linked to reduced enzyme activity and elevated retinoid levels and not increased enzyme activity. Compared to 13-trans, 13-cis is preferentially sequestered in the nucleus of embryonic cells, where it is subsequently converted to 13-trans, another teratogen. It is proposed that measuring (1) maternal CYP26A1 and CYP26B1 activities and 13-cis concentrations in the blood of pregnant mothers, and (2) monitoring total maternal intake of beta carotene and preformed vitamin A, could reduce the incidence of neurodevelopmental disorders. Methods for measuring maternal CYP26A1, CYP26B1, and 13-cis in plasma and blood are described.

### DETAILED DESCRIPTION OF THE INVENTION

13-cis is a potent teratogen known to cause abnormalities in the hindbrain, including the cerebellum, as observed in imaging studies of developing primate embryos. It exists in dynamic equilibrium with 13-trans, and both forms cross the placental barrier to enter fetal circulation. In humans, retinoic acid levels are tightly regulated, and both excess and deficiency are associated with CNS abnormalities in the fetus. In pregnant mothers the plasma 13-cis concentration is highest in the first 8-12 weeks of pregnancy and declines thereafter by 0.04 nM per week. Neurocognitive impairment even in the absence of any physical defects has been reported to be 30%-60% of children exposed to 13-cis during the prenatal period.

In pregnant mothers, BCMO1 regulates the conversion of beta carotene to retinoids. BCO1 is a cytosolic enzyme that catalyzes the central cleavage of beta-carotene at the 15,15′double bond, producing two molecules of retinal (vitamin A aldehyde). This step is essential for converting dietary beta-carotene (provitamin A) into vitamin A, which is necessary for vision, immune function, and cellular differentiation. The conversion of retinol to retinoic acid involves retinol dehydrogenases and alcohol dehydrogenases for the initial step, followed by retinaldehyde dehydrogenases for the irreversible conversion. BCO1 is primarily located in cells, with its activity confined to the cytosol, where it plays a critical role in vitamin A metabolism.

Umbilical cord blood, including both arteries and vein, contains beta carotene at concentrations approximately twenty times lower than those found in maternal blood. However, separate measurements for the arteries and vein have not been reported in the literature and could be very helpful.

If beta carotene concentration is lower in the umbilical arteries than in the vein, it implies that the fetus metabolizes or stores beta carotene as blood circulates through fetal tissues before returning to the placenta. If concentrations are similar, it suggests minimal fetal metabolism or storage, implying that the placenta primarily regulates beta carotene transfer, and the fetus does not significantly alter beta carotene levels during circulation. Furthermore, if a mother with a normal BCO1 phenotype consumes large amounts of beta carotene and preformed vitamin A from foods and prenatal vitamins, and has decreased CYP26A1 and/or CYP26B1 activity, maternal retinoid levels may rise, potentially increasing fetal exposure.

### Inhibitors of CYP26A1 and CYP26B1

CYP26A1 is present in small amounts in human plasma, while CYP26B1 is found primarily in cells. Accurate differentiation of cytochrome activity may require immunoprecipitation, especially if cell lysis occurs in blood samples. Alternatively, the CYP26A1 inhibitor 3-{4-[2-(5,5,8,8-tetramethyl-5,6,7,8-tetrahydronaphthalen-2-yl)-1,3-dioxolan-2-yl]phenyl}-4-propanoic acid, which has a 43-fold selectivity for CYP26A1 over CYP26B1 (IC₅₀=340 nM), can be used to selectively inhibit CYP26A1 and distinguish its activity from CYP26B1. To differentiate CYP26B1 activity from other retinoid-metabolizing enzymes, a CYP26B1-selective inhibitor such as DX314 can be added to parallel reactions.

### Genetic Variability of CYP26A1 and CYP26B1

Numerous genetic variants of CYP26A1 have been identified across different populations. Notable single nucleotide polymorphisms (SNPs) include F186L (558C>A), which results in a phenylalanine to leucine substitution at position 186 and is found in Caucasians; R173S (517C>A), an arginine to serine substitution at position 173 observed in African-Americans; and C358R (1072T>C), a cysteine to arginine substitution at position 358 present in Asians. Functional studies indicate that the F186L and C358R variants significantly decrease the metabolism of 13-trans, while R173S does not alter metabolism compared to the wildtype. Additional variants include missense mutations, transcript variants, and splice site changes. Most of these variants reduce CYP26A1 activity, and none are known to increase it.

Pathogenic variants in CYP26B1 have been identified in individuals exhibiting a broad spectrum of clinical features, ranging from severe conditions such as skull anomalies, craniosynostosis, encephalocele, radio-humeral fusion, oligodactyly, and a narrow thorax, to milder presentations including craniofacial dysmorphism, restricted joint mobility, hearing loss, and intellectual disability. Documented genetic changes include missense mutations, compound heterozygous variants, and synonymous variants. For example, the p. (Pro118Leu) and p. (Arg234Gln) mutations were found in a family with mild craniofacial and skeletal abnormalities. Functional studies demonstrated that these variants led to a partial reduction in retinoic acid metabolism (1.7× and 2.3× less than wild-type). Importantly, all identified variants are associated with decreased, rather than increased, CYP26B1 activity and generally increased plasma retinoids.

### Measurements of CYP26A1, CYP26B1, and 13-Cis

### Protocol for Measurement of CYP26A1 Activity in Maternal Blood (Plasma)

### 1. Sample Collection

Maternal blood is collected via standard venipuncture into anticoagulant-treated tubes (e.g., EDTA) to prevent clotting. Samples are maintained at 4° C. until processing.

### 2. Plasma Preparation

Blood samples are centrifuged at approximately 1,500×g for 10 minutes at 4° C. The plasma fraction is carefully separated and transferred to clean tubes, ensuring minimal cellular contamination.

### 3. Substrate Preparation

A solution of retinoic acid, the substrate for CYP26A1, is prepared at a known concentration (typically micromolar) in an appropriate buffer such as phosphate-buffered saline.

### 4. Enzymatic Incubation

Aliquots of maternal plasma are incubated with the retinoic acid substrate at 37° C. for a predetermined period (e.g., 30-60 minutes). Control incubations include plasma without substrate and substrate without plasma.

### 5. Reaction Termination

The enzymatic reaction is terminated by the addition of ice-cold methanol or acetonitrile. Samples are vortexed and centrifuged to precipitate proteins.

### 6. Metabolite Extraction

The supernatant containing retinoid metabolites is collected. If necessary, solid-phase extraction is performed to further purify the metabolites.

### 7. Analytical Quantification

Metabolites are analyzed using high-performance liquid chromatography (HPLC) or liquid chromatography-mass spectrometry (LC-MS). The conversion of retinoic acid to its primary metabolite, 4-hydroxy-retinoic acid, is quantified as a measure of CYP26A1 activity.

### 8. Data Analysis

Enzyme activity is calculated based on the amount of metabolite formed per unit time and per volume of plasma. Results are compared to reference standards and controls.

### 9. Interpretation

Reduced CYP26A1 activity may suggest genetic variability or regulatory impairment. Genetic analysis of CYP26A1 may be performed in parallel for comprehensive assessment.

### Protocol for Measurement of CYP26B1 Activity in Maternal Blood Samples (With Cell Lysis)

### 1. Sample Collection

Collect maternal blood via standard venipuncture into anticoagulant-treated tubes (e.g., EDTA) to prevent clotting. Maintain samples at 4° C. until processing.

### 2. Cell Isolation

Centrifuge blood samples at approximately 1,500×g for 10 minutes at 4° C. to separate plasma and cellular fractions. Carefully collect the cellular pellet (containing leukocytes and other nucleated cells) for analysis.

### 3. Cell Lysis

Resuspend the cellular pellet in an appropriate lysis buffer (e.g., phosphate-buffered saline with 0.1% Triton X-100 or other non-denaturing detergent). Incubate on ice for 15-30 minutes, vortexing periodically to ensure complete lysis. Optional: Use mechanical disruption (e.g., sonication) for enhanced lysis.

### 4. Lysate Preparation

Centrifuge the lysed cell suspension at 10,000×g for 10 minutes at 4° C. to remove cellular debris. Collect the supernatant, which contains intracellular proteins including CYP26B1.

### 5. Substrate Preparation

Prepare a solution of retinoic acid (the substrate for CYP26B1) at a known concentration (typically micromolar) in an appropriate buffer.

### 6. Enzymatic Incubation

Incubate aliquots of the cell lysate with retinoic acid substrate at 37° C. for a predetermined period (e.g., 30-60 minutes). Include control incubations: lysate without substrate and substrate without lysate.

### 7. Selective Inhibition (Optional)

To distinguish CYP26B1 activity from other retinoid-metabolizing enzymes, add a CYP26B1-selective inhibitor such as 3-[4-[(3-adamantan-1-yl-4-hydroxyphenyl)amino]phenyl]propanoic acid (DX314) to parallel reactions. This allows for specific measurement of CYP26B1-dependent metabolism.

### 8. Reaction Termination

Terminate the enzymatic reaction by adding ice-cold methanol or acetonitrile. Vortex and centrifuge to precipitate proteins.

### 9. Metabolite Extraction

Collect the supernatant containing retinoid metabolites. If necessary, perform solid-phase extraction to further purify metabolites.

### 10. Analytical Quantification

Analyze metabolites using high-performance liquid chromatography (HPLC) or liquid chromatography-mass spectrometry (LC-MS). Quantify the conversion of retinoic acid to its metabolites (e.g., 4-hydroxy-retinoic acid) as a measure of CYP26B1 activity.

### 11. Data Analysis

Calculate enzyme activity based on the amount of metabolite formed per unit time and per volume of lysate. Compare results to reference standards and controls.

### 12. Interpretation

Reduced CYP26B1 activity may suggest genetic variability or regulatory impairment. Genetic analysis of CYP26B1 may be performed in parallel for comprehensive assessment.
This protocol ensures accurate measurement of CYP26B1 activity by lysing cells to access the intracellular enzyme, followed by substrate incubation and analytical quantification.
   Protocol for Measurement of CYP26A1 Activity in Maternal Blood Samples (with Cell Lysis)

### 1. Sample Collection

Collect maternal blood via standard venipuncture into anticoagulant-treated tubes (e.g., EDTA) to prevent clotting. Maintain samples at 4° C. until processing.

### 2. Cell Isolation

Centrifuge blood samples at approximately 1,500×g for 10 minutes at 4° C. to separate plasma and cellular fractions. Carefully collect the cellular pellet (containing leukocytes and other nucleated cells) for analysis.

### 3. Cell Lysis

Resuspend the cellular pellet in an appropriate lysis buffer (e.g., phosphate-buffered saline with 0.1% Triton X-100 or other non-denaturing detergent). Incubate on ice for 15-30 minutes, vortexing periodically to ensure complete lysis. Optional: Use mechanical disruption (e.g., sonication) for enhanced lysis.

### 4. Lysate Preparation

Centrifuge the lysed cell suspension at 10,000×g for 10 minutes at 4° C. to remove cellular debris. Collect the supernatant, which contains intracellular proteins including CYP26A1.

### 5. Substrate Preparation

Prepare a solution of retinoic acid (the substrate for CYP26A1) at a known concentration (typically micromolar) in an appropriate buffer.

### 6. Enzymatic Incubation

Incubate aliquots of the cell lysate with retinoic acid substrate at 37° C. for a predetermined period (e.g., 30-60 minutes). Include control incubations: lysate without substrate and substrate without lysate.

### 7. Selective Inhibition (Optional)

To distinguish CYP26A1 activity from other retinoid-metabolizing enzymes, add a CYP26A1-selective inhibitor such as the CYP26A1 inhibitor 3-{4-[2-(5,5,8,8-tetramethyl-5,6,7,8-tetrahydronaphthalen-2-yl)-1,3-dioxolan-2-yl]phenyl}4-propanoic acid, which has a 43-fold selectivity for CYP26A1 over CYP26B1 and can be used to selectively inhibit CYP26A1 and distinguish its activity from CYP26B1 to parallel reactions. This allows for specific measurement of CYP26A1-dependent metabolism.

### 8. Reaction Termination

Terminate the enzymatic reaction by adding ice-cold methanol or acetonitrile. Vortex and centrifuge to precipitate proteins.

### 9. Metabolite Extraction

Collect the supernatant containing retinoid metabolites. If necessary, perform solid-phase extraction to further purify metabolites.

### 10. Analytical Quantification

Analyze metabolites using high-performance liquid chromatography (HPLC) or liquid chromatography-mass spectrometry (LC-MS). Quantify the conversion of retinoic acid to its metabolites (e.g., 4-hydroxy-retinoic acid) as a measure of CYP26A1 activity.

### 11. Data Analysis

Calculate enzyme activity based on the amount of metabolite formed per unit time and per volume of lysate. Compare results to reference standards and controls.

### 12. Interpretation

Reduced CYP26A1 activity may suggest genetic variability or regulatory impairment. Genetic analysis of CYP26A1 may be performed in parallel for comprehensive assessment.
This protocol ensures accurate measurement of CYP26A1 activity by lysing cells to access the intracellular enzyme, followed by substrate incubation and analytical quantification

### Protocol for Measurement of 13-Cis in Maternal Blood

### 1. Sample Collection

Maternal blood is collected via standard venipuncture into anticoagulant-treated tubes (e.g., EDTA) to prevent clotting. Samples are maintained at 4° C. until processing.

### 2. Plasma Preparation

Blood samples are centrifuged at approximately 1,500×g for 10 minutes at 4° C. The plasma fraction is carefully separated and transferred to clean tubes, ensuring minimal cellular contamination.

### 3. Extraction of Retinoids

Plasma samples are mixed with ice-cold methanol or acetonitrile to precipitate proteins and extract retinoids. The mixture is vortexed and centrifuged, and the supernatant containing retinoids is collected. If necessary, solid-phase extraction is performed to further purify retinoids.

### 4. Chromatographic Separation

The extracted retinoids are subjected to high-performance liquid chromatography (HPLC) or liquid chromatography-mass spectrometry (LC-MS) for separation. Chromatographic conditions are optimized to resolve 13-cis retinoic acid from other retinoid isomers.

### 5. Quantification

13-cis is quantified by comparison to reference standards using HPLC or LC-MS. Calibration curves are prepared with known concentrations of 13-cis retinoic acid to ensure accurate quantification.

### 6. Data Analysis

The concentration of 13-cis in maternal plasma is calculated based on the chromatographic peak area or mass spectrometric signal, referenced to calibration standards. Results are compared to controls and reference ranges.

### 7. Interpretation

Elevated levels of 13-cis may indicate increased maternal and/or fetal exposure associated with increased nuclear concentration of 13-cis and 13-trans.
This protocol provides a reproducible method for the measurement of 13-cis in maternal plasma, supporting claims related to the diagnosis and study of neurodevelopmental disorders.

### REFERENCES

### Non-Patent Literature

Goldberg, J.S., Monitoring Maternal Beta Carotene and Retinol Consumption May Decrease the Incidence of Neurodevelopmental Disorders in Offspring, Clinical Medicine Insights: Reproductive Health, 6, 1-8, 2012.
Choi JS, Koren G, Nulman I. Pregnancy and isotretinoin therapy. CMAJ. 2013; 185(5):411-413. doi:10.1503/cmaj.120729.
Jeong H, Armstrong AT, Isoherranen N, Czuba L, Yang A, Zumpf K, et al. Temporal Changes in the Systemic Concentrations of Retinoids in Pregnant and Postpartum Women. PLOS ONE, 18(2): e0280424, 2023.
Teerlink, Tom et al. Simultaneous analysis of retinol, all-trans- and 13-cis-retinoic acid and 13-cis-4-oxoretinoic acid in plasma by liquid chromatography. Journal of Chromatography B, 694 (1997), pp. 307-313.
Topletz, A.R., Zhong, G., Maeda, A., Nakamura, K., Osawa, M., and Isoherranen, N. CYP26A1: A major retinoic acid-metabolizing enzyme in human liver. Journal of Biological Chemistry, vol. 287, no. 8, 2012, pp. 6536-6548. doi:10.1074/jbc.M111.315382.
Laue, K., Janicke, M., Plaster, N., et al. Identification and characterization of the human retinoic acid-metabolizing enzyme CYP26B1. Journal of Biological Chemistry, vol. 278, no. 36, 2003, pp. 33396-33403. doi:10.1074/jbc.M303938200.
Silveira KC, Fonseca IC, Oborn C, et al. CYP26B1-related disorder: expanding the ends of the spectrum through clinical and molecular evidence. Human Genetics. 2023 Sep 27. doi:10.1007/s00439-023-02598-2.
Lee, S.A., Taimi, M., Helvig, C., et al. Functional characterization of genetic variants in the CYP26A1 gene in humans. OMIM Entry 602239.

Having described my invention, I claim:

1. A method for diagnosing a neurodevelopmental disorder in a human subject comprising: measuring the activity of CYP26A1 in a maternal plasma sample.

2. A method for diagnosing a neurodevelopmental disorder in a human subject comprising: measuring the concentration of 13-cis retinoic acid in a maternal plasma sample.

3. A method for diagnosing a neurodevelopmental disorder in a human subject comprising: measuring the activity of CYP26B1 in a maternal blood sample.

4. The method of claim 3, wherein the enzyme measured is selected
from the group consisting of CYP26B1 and CYP26A1.
